(19) United States
(12) Patent Application Publication
(10) Pub. No.: US 2008/0032306 A1
(43) Pub. Date: Feb. 7, 2008

(54) METHODS AND KITS FOR DETECTION OF NUCLEIC ACID AMPLIFICATION PRODUCTS

(76) Inventor: Joseph A. Sorge, Wilson, WY (US)

Correspondence Address: AGILENT TECHOLOGIES INC, P.O BOX 7599, BLDG E , LEGAL, LOVELAND, CO, 80537-0599, US

(21) Appl. No.: 11/834,997

(22) Filed: Aug. 7, 2007

Related U.S. Application Data
(60) Provisional application No. 60/836,435, filed on Aug. 7, 2006.

(51) Int. Cl. C12Q 1/68; C12M 1/00

(52) U.S. Cl. 435/6; 435/287.2

(57) ABSTRACT

The present invention provides methods, products, and kits for detection of target nucleic acids. Detection is based, at least in part, on detection of chromophores that have different chromogenic properties depending on their association with double-stranded nucleic acids. In general, labeled reagents are exposed to denatured nucleic acids, and the nucleic acids are permitted to renature. Incorporation of the chromogenic labels into the renaturing nucleic acids produces a detectable signal, which can be correlated to the presence and amount of target nucleic acid in the sample. The method is particularly suitable for detection of in vitro amplification products.

### CROSS-REFERENCE TO RELATED APPLICATIONS

The present application relies on the disclosure of and claims the benefit of the filing date of U.S. provisional patent application No. 60/836,435, filed 7 Aug. 2006, the entire disclosure of which is hereby incorporated herein by reference.

### BACKGROUND OF THE INVENTION

1. Field of the Invention

The present invention relates to the field of molecular biology. More specifically, the invention relates to processes for detecting nucleic acids resulting from amplification reactions, such as, but not limited to, the Polymerase Chain Reaction (PCR) and its various derivative processes (e.g., RT-PCR, QPCR, RT-QPCR, etc.).

2. Description of Related Art

The power of molecular biology techniques to identify and characterize samples containing biological material is well documented. Techniques for identifying nucleic acids in many different types of samples from many different sources have been developed and are being used daily in research settings, clinical investigations, crime investigations, and agriculture. The techniques often rely on identification of nucleic acid sequences in a sample, and correlating those sequences with a source organism.

PCR is a useful analytical tool that utilizes basic principles of molecular biology, and applies to all the fields of analysis where nucleic acids play a direct or indirect role. In principle, PCR analytic techniques (see, for example, R. K. Saiki et al. Science 239:487-491 (1988)) and other enzymatic amplification techniques (see, for example, J. C. Guatelli et al., Proc. Natl. Acad. Sci. 87:1874-1878 (1990)) allow the detection of small amounts of DNA or RNA in an aqueous solution. Numerous derivative assays based on PCR have been developed to amplify various starting materials, and the details of those assays are well documented in the literature.

Among the many techniques known in the art, WO 91/02815 describes the detection of specific DNA and RNA from biological sample material using a DNA/RNA amplification method in combination with a detection technique, such as temperature gradient gel electrophoresis. Likewise, others have used multiple distinct techniques for amplification and detection of target nucleic acids (see, for example, K. Henco and M. Heibey, Nucleic Acids Res. 19:6733-6734 (1990); J. Kang et al., Biotech. Forum Europe 8:590-593, (1991); and G. Gilliland et al., Proc. Natl. Acad. Sci. 87:2725-2729 (1990)).

One commonly employed technique for detecting target nucleic acids is practiced using the TaqMan® technology of Roche Molecular Systems, Inc. TaqMan® probes are nucleic acid-based probes comprising a fluorophore on one end and a quencher on the other. Typically, the probes are 18-22 bases in length, a length that is short enough to allow the quencher to quench the fluorescence of the fluorophore. In use, the probe binds to a target sequence, which is a template for amplification. As the polymerase moves down the template strand during an amplification reaction, it encounters the probe. The 5′ exonuclease activity of the polymerase digests the probe, releasing the fluorophore from the molecule. The free fluorophore is now able to generate an unquenched signal, which can be detected by an appropriate detector. The increase in fluorophore signal can be correlated to the amount of amplified template. Similar technologies include Molecular Beacons and Scorpions.

Another widely used technology for detecting amplified nucleic acids relies on the SYBR Green series of dyes. This technology is based on differential emission of a detectable signal of the dye when free in solution as compared to bound to a nucleic acid. More specifically, SYBR Green dyes are sequence non-specific dyes that have an increased emission level (approximately 1000-fold higher) when bound to double-stranded DNA as compared to when free in solution. The signal intensity of a sample can be monitored and correlated to the amount of DNA in a sample (e.g., an amplification reaction).

Although numerous solutions for amplification and detection of target nucleic acid sequences have been devised, there still exists a need in the art for techniques that are rapid and easy to use. In addition, techniques that minimize the amount of reagent handling, and thus sample-to-sample variation, are desirable.

### SUMMARY OF THE INVENTION

The present invention addresses needs in the art by providing methods and products for amplification and detection of nucleic acid sequences of interest. The invention provides a process and product for characterizing one or more nucleic acid complexes, allowing for the detection or analysis of one or more one in vitro amplified nucleic acids, either inside or outside an amplification reaction chamber. In addition, the invention encompasses a process and product for the qualitative and quantitative analysis of at least one in vitro amplified nucleic acid product in a reaction chamber.

In a first aspect, a process or method for detecting a nucleic acid of interest (also referred to herein as a “target”), is provided, In general, the process comprises: forming a composition comprising one or more nucleic acids, such as those formed as products of nucleic acid amplification reactions, and one or more detectable labels, under conditions where at least some of the nucleic acids exist in a single-stranded state; decreasing the temperature of the composition to allow the detectable label(s) and nucleic acid(s) to stably contact each other to form complexes; and detecting the complexes, wherein detection of complexes is indicative of the presence of the nucleic acid of interest. Typically, the method is practiced, at least in part, in vitro. In addition, typically, the method is practiced on samples comprising one or more amplification products of an in vitro amplification process, such as PCR. Unlike solutions devised previously, the present method for detection of target sequences can be performed in a single vessel or container, without the need to transfer the complexes from a reaction vessel to a detection vessel or an apparatus for detection.

In another aspect, the invention provides articles of manufacture for performing nucleic acid amplification and detection methods. In general, the articles of manufacture comprise one or more reaction vessels for performing amplification reactions, detection assays, or both. The articles comprise at least one wall that is suitable for transmission of a detectable signal to an external detector, such as an instrument that can detect electromagnetic radiation, particularly in the ultraviolet (UV) and visible (VIS) ranges (i.e., a UV-VIS detector). While not limited in shape, size, or manner of construction, exemplary articles of manufacture according to the invention are multi-well microtiter plates, micro-centrifuge tubes (e.g., PCR reaction tubes), and relatively thin plastic sheets comprising multiple reaction/detection wells. It is to be understood that the articles of manufacture are not to be construed so broadly as to encompass apparatuses for separation of nucleic acids, such as gel electrophoresis units, but rather are focused on well-defined vessels suitable for performing chemical and biochemical reactions.

In yet another aspect, the invention comprises kits for amplification and/or detection of target nucleic acids. In general, the kits comprise at least one article of manufacture according to the invention, in packaged form. The kits may also comprise some or all of the reagents, solutions, etc. needed to perform a method according to the invention. As detailed below, in some embodiments, the kits of the invention comprise all of the reagents needed to detect a target nucleic acid sequence, and use of the kits merely requires addition of a sample suspected of containing the target nucleic acid.

### DETAILED DESCRIPTION OF VARIOUS EMBODIMENTS OF THE INVENTION

Reference will now be made in detail to various exemplary embodiments of the invention. The following detailed description is provided to assist the reader in understanding certain details and embodiments of aspects of the invention, and is not to be understood as a limitation on the scope or content of the invention.

In a first aspect, a process or method for detecting a target nucleic acid is provided. In many embodiments, the process comprises: forming a composition comprising one or more nucleic acid amplification products present in an amplification reaction mixture and one or more detectable labels, under conditions where at least some of the amplification product(s) exist in a single-stranded state; decreasing the temperature of the composition to allow the detectable label(s) and amplification product(s) to stably contact each other to form complexes; and detecting the complexes, wherein detection of complexes is indicative of the presence of the amplification product of interest. The methods described herein in exemplary embodiments encompass the use of a cooling curve to identify and/or characterize nucleic acids originally present in a sample, and/or to detect nucleic acids produced in an in vitro molecular reaction such as PCR or cDNA synthesis. The target nucleic acids are detected by incorporation of the label(s) into the renaturing or renatured target, and detection of the label(s). The labels can be used directly as reagents or can comprise part of a probe that includes a nucleic acid portion. The renaturation of denatured target nucleic acids during the course of a cooling curve is monitored in some instances with intercalating dyes (e.g., ethidium bromide or thiazole orange dyes) on a molecular level, where the dyes are not fixed to a nucleic acid probe. These dyes intercalate under native conditions between adjacent base pairs in double-stranded DNA or RNA. In an intercalated condition, the fluorescence yield increases up to 20 fold and the lifetime of the excited state about 10 fold. When such system is subjected to a decreasing thermal gradient, the thermodynamically most stable regions of the nucleic acid helix begin to renature initially. Mispairing as generated in heteroduplex formation destabilizes the corresponding sequence region and results in delayed renaturation of the latter. Dye molecules (such as ethidium bromide) initially unbound, will be intercalated in the double stranded region(s) that renature, resulting in an increase of the overall fluorescence signal. In one embodiment, the dye concentration is selected such that free dye and bound dye are in thermodynamic equilibrium and free dye is present in significant excess. Only at lower temperatures will the corresponding sequence region of a heteroduplex relative to a homoduplex, also renature, giving rise to further stepwise increase of the fluorescence signal. From the intensity ratio of both steps, the relative ratio of amounts of homoduplex and heteroduplex can be determined.

According to the method of the invention, the target nucleic acid is not limited in sequence, source, or length. It thus can be any nucleic acid from any organism or it can be an artificially generated sequence. Accordingly, it may be from a prokaryote, eukaryote, or virus, or may be an artificial sequence added to a sample, such as a control sequence for confirming successful practice of the method. It likewise may be a genetically engineered sequence found in a genetically modified organism (GMO), found in a laboratory/research organism, or in an organism found in the environment. It may be single-stranded or double-stranded. The length of the target nucleic acid is not critical to practice of the invention; thus, for example, the process may be practiced on a nucleic acid target that is relatively small, for example on the order of tens or hundreds of bases in length, or may be practiced on a nucleic acid target that is relatively large, for example on the order of millions of bases in length. It is to be understood that the term “target” is a term that denotes an intent of a practitioner, and thus can refer to an entire nucleic acid or a portion of it. That is, the method of the present invention may be practiced to detect a target sequence of any length, where that sequence may be the entire sequence of a nucleic acid employed in the method or may be only a portion of the entire sequence of the nucleic acid employed in the method. The particular length and sequence of the target will not substantially affect practice of the invention, and thus the method of the invention does not rely on any particular sequences. Those of skill in the art are fully capable of designing probes and primers that are suitable for detection of any target nucleic acid of interest.

In situations where the method is practiced to detect a target having one or more known nucleotide sequences, the method can be performed using probes and primers (discussed below in more detail) that are specifically tailored for those known sequences. Likewise, where the method is practiced to detect one or more organisms having a homologous (but not necessarily identical) target sequence, the method can be performed using probes and primers that are tailored to detect all sequences having the homologous target sequence (e.g., using a mixture of probes or conditions that allow for binding and detection of a probe to homologous but not perfectly identical, sequences. On the other hand, where the target sequence is one that is unknown (e.g., where the method is practiced to determine if any nucleic acids are present in a sample) a collection of primers and/or probes with random sequences may be used.

The target nucleic acid can be found in any type of sample and in any state of purity that is suitable for detection by the method of the invention. The sample may thus be, for example, an aqueous sample comprising purified (at least to some extent) nucleic acid, an environmental sample comprising a complex mixture of substances, and the like. Preferably, the nucleic acid is present in an in vitro amplification reaction mixture.

According to embodiments of the method of the invention, a composition is formed that comprises one or more nucleic acid amplification products present in an amplification reaction mixture and one or more detectable labels. Preferably, the composition is an aqueous composition. In general, forming a composition comprises any action that results in creation of a composition that comprises the nucleic acid(s) and detectable label(s). It thus may comprise combining two or more compositions, such as by adding one composition to another. Alternatively, it can comprise adding water or an aqueous composition to a solid composition and allowing the water/aqueous composition to dissolve some or all of the substances in the solid composition. For example, it can comprise adding water or an aqueous buffer solution to a lyophilized (freeze-dried) composition and providing conditions that allow the water/buffer to hydrate and/or dissolve the lyophilized composition. Forming a composition can result from active actions of the practitioner (e.g., mechanical disruption, mixing, etc.) or by passive actions (e.g., adding water and allowing sufficient time for formation of a composition). Preferably, the composition that is formed is a homogeneous composition. In preferred embodiments, the composition comprises all of the components of a nucleic acid amplification reaction, to which is added one or more detectable labels, and, optionally, other substances, which might or might not affect signal production.

The method of the invention detects target nucleic acids. In some embodiments, it is directed to detecting double-stranded nucleic acid sequences by including a detectable label in the double-stranded nucleic acid. As detailed below, the label may be incorporated into the target when the target is in a double-stranded state or during formation of a double-stranded state. In situations where incorporation occurs during formation of a double-stranded state, it is preferred that the target be provided in a single-stranded state. For example, where detection is by way of detecting an amplification product, it is preferred that the detectable label be present under conditions where at least some of the amplification product(s) exist in a single-stranded state, such as at the end of the amplification cycling process and at a relatively high temperature, which causes denaturation of most or all of the amplification products. Of course, other means for providing single-stranded target nucleic acid may be used, such as by adjusting of salt concentrations, pH, inclusion of denaturants, and combinations of these and other techniques known in the art.

Where the method of the invention is practiced using nucleic acid amplification products as targets, the composition will typically be formed at, or soon thereafter be subjected to, a temperature that is sufficient to denature some or all of the double-stranded nucleic acids in the sample. While the temperature will vary to some extent depending on the size and nucleotide sequence of the target nucleic acid, in general, a temperature of at least 80° C. will be used. For example, a temperature of 85° C., 90° C., 94° C., or 98° C., or higher may be used. It is to be noted at this point that, throughout this document, where a specific range of values are recited, all values within the range are to be understood as being recited as well. For example, where a range of values from 80° C. to 100° C. is discussed, it is to be understood that each and every specific value within that range is also recited. Those of skill in the art will immediately recognize each value, including fractions thereof, within the range without the need for specific recitation of each value herein.

According to embodiments of the method, the temperature of the composition is reduced or decreased to allow for renaturation (e.g., hydrogen bonding) of the target nucleic acid. During this process, one or more detectable labels may be incorporated into the renaturing/renatured nucleic acids, and it is this incorporation that allows for detection of the target nucleic acid(s). While not being limited to any particular starting and ending temperatures, in general, the temperature of the composition is reduced from 80° C. or above to 60° C. or below, such as from 95° C. to room temperature (21° C. to 25° C.). Of course, the practitioner may select any desired values for starting and stopping the temperature gradient. The temperature reduction process may be performed at any suitable rate, but is typically performed at a rate that allows for real-time monitoring of renaturation by incorporation of label into the target nucleic acid and for proper renaturation of nucleic acid strands. The cooling gradient may be performed as a continuous gradient or in a step-wise fashion, and may be paused at any time, according to the practitioner's desires.

In one embodiment of the process described herein, the gradient can be a continuous gradient with respect to the rate of decrease in temperature over time. In one embodiment, the continuous gradient is linear, though non-linear gradients are also encompassed by the gradients described herein. The term “continuous temperature gradient” is a gradient in which the temperature is changing (i.e., increasing or decreasing) continuously with respect to a specified time interval, (preferably decreasing with respect to the specified time interval). The term “stepwise gradient” is a gradient in which the temperature is changing (i.e., increasing or decreasing) over a given time interval, but the change is fragmented into a series of steps, each step comprising two time periods: a first time period in which the temperature changes (preferably decreases), and a second time period in which the temperature is maintained. In a preferred embodiment, the steps are of equal length. In another embodiment, the steps are of unequal lengths.

In one embodiment, the total course of the entire temperature gradient is composed of one or more continuous gradients and/or one or more stepwise gradients. If the total course of the entire temperature gradient contains multiple continuous gradients, one or more or all or none of the multiple continuous gradients are identical. If the total course of the entire temperature gradient contains multiple stepwise gradients, one or more or all or none of the multiple continuous gradients are identical. Each of the multiple gradients can be arranged in any order within the entire temperature gradient.

As mentioned, the gradient can be a stepwise gradient with respect to the rate of decrease in temperature over time. The time intervals for each degree of temperature decrease over time in a stepwise cooling gradient can range from about 1 second to about 1500 seconds or more (e.g., 30 minutes, 60 minutes). Alternatively, the gradient can have both a stepwise component and a linear component. Whether the gradient is a continuous or step-wise gradient, it will typically be completed within 30 minutes to allow for the method to be performed conveniently and relatively rapidly.

In embodiments of the method where the temperature of the composition is lowered, decreasing the temperature of the composition allows the detectable label(s) and amplification product(s) to stably contact each other to form complexes. In forming the composition, target nucleic acid and label are combined; however, a stable interaction between the two (where appropriate) might not occur at the temperature provided. Lowering the temperature allows for productive interaction of the two, resulting in generation of a detectable signal that can be correlated with the presence of the nucleic acid (e.g., amplification product) of interest. Detection is based on detection of a signal emitted from a labeled substance, which can be part of a probe. The labeled probe can be detected by any suitable means, but is typically detected using spectroscopic techniques known and widely used in the art.

As mentioned above, the probe may be a detectable substance that functions as a stand-alone unit or may be a multi-part structure that includes a nucleic acid portion and a label portion. Thus, for multi-part probes, the probe, the parameter of which is to be detectable spectroscopically, preferably contains at least one labeled residue, for example a fluorescent residue, and preferably has intercalating properties, and a nucleic acid component. In this way, the nucleic acid component can be used to generate specificity of the probe, while the label can be used for spectroscopic detection. In a preferred mechanism of action, the label shows different spectroscopic characteristics when alone in solution and when present in a complex with other substances. The interaction of the probe with the target nucleic acid, such as for example, in vitro amplified nucleic acid, as a function of its denaturation condition, can thus be accompanied by a change in the spectroscopically measured signal of the probe. This, for example, may take place by intercalation of a dye moiety into the nucleic acid double helix or by dilution or concentration effects within the measuring compartment.

The terms “oligonucleotide” and “oligo”, as used herein in referring to the probe of the present invention, are defined as molecules comprising about 12 or more nucleotides, preferably more than about 24, and more preferably about 36 nucleotides. The exact size will depend upon many factors which, in turn, depend upon the ultimate function and use of the oligonucleotide. Preferably, an oligonucleotide that functions as an extension primer will be sufficiently long to prime the synthesis of extension products in the presence of a catalyst, e.g., DNA polymerase, and deoxynucleotide triphosphates. The exact lengths of the primer will depend on many factors, including temperature, source of primer, and use of the method. In diagnostic applications, for example, the oligonucleotide primer typically contains 15-25 or more nucleotides, depending on the complexity of the target sequence. For non-extension product applications, the oligonucleotide generally contains between 10 and 25 nucleotides. Shorter oligonucleotides generally require cooler temperatures to form sufficiently stable hybrid complexes with template (as known in the art).

The term “probe” as used herein is used generally to denote a substance that can be used to detect a nucleic acid of interest. It thus comprises, at the least, a detectable label. In embodiments, it is an oligonucleotide or polynucleotide that hybridizes to a target nucleic acid, such as a target in a sample, resulting in a detectable signal, such as one having attached to it one or more intercalating dyes. In other embodiments, it is a dye that intercalates into double stranded polynucleotides. In yet a further embodiment, either the probe or the target can be labeled with one or more than one label, and interaction of the probe and target produces a detectable signal or a change in a previously occurring detectable signal. Labels for use in the present invention are well known to those of skill in the art, and include, for example, intercalating dyes, radioactive labels, fluorescent labels, and the like.

In a preferred embodiment, the oligonucleotide primers for amplification reactions contain a spacer-linked fluorescent dye capable of intercalating if the primer is located in a double-helical region. Upon thermal denaturation of the double-helix, the fluorescent properties of the dye are modified (for example, as described in Thuong, N. T. and Chassignol, M., Tetrahedron Letters 28:4157-4160 (1987); Thuong, N. T. et al., Proc. Natl. Acad. Sci. U.S.A. 84:5129-5133 (1987); Helene, C., in “DNA-Ligand Interactions”, Plenum Publishing Corporation, 127-140 (1987); W. Gushelbauer and W. Saenger, Ed.).

The method of the invention is useful for detection of any nucleic acid of interest in a rapid, simple format that can be used in conjunction with multiple commercially available detection devices. The method can be used in any type of setting, from research to clinical diagnostics to environmental testing. For example, it can be used to detect one or more organisms (including viruses) in a sample taken from a patient suffering from an infection or other disease or disorder, taken from an environmental sample, or taken from a food sample in the food distribution chain. In one application, detection of the presence of nucleic acid from an infectious agent in a clinical sample can be accomplished, allowing for diagnosis or confirmation of the cause of a disease or disorder and implementation of an appropriate treatment regimen. In another application, detection of the presence of nucleic acid from a disease-causing organism in an environmental sample can be accomplished, allowing for remedial action to be taken to protect plants and animals (including humans) from the organism. Yet again, detection of a target nucleic acid in a research sample can allow for confirmation of the presence of an organism, as a research target or as a contaminant. With respect to the food supply, detection of a target sequence can indicate the presence of an organism in the food supply, which may be dangerous (e.g., fungal contamination) or simply undesirable (e.g., GMO material in “organically grown” foods). Indeed, the present method is suitable for use in any situation where detection of organisms (including plants and animals) is desired. Non-limiting examples of particular sources include: live tissue, dead tissue, fossil tissue, as well as tissue which no longer displays active metabolism in vivo, or from body fluids, from in vitro cell cultures, or from environmental samples. The process disclosed herein allows for qualitative and quantitative detection of cellular genes and genes of infectious pathogens directly or via their RNA gene products as a wild type sequence or as variants. Using the process disclosed herein it is possible, for example, to detect mutations, point mutations, deletions, insertions, and rearrangements within a DNA/RNA nucleic acid. Using the quantitative analysis, it is also possible to determine the concentrations of such changes in the nucleic acid.

Moreover, the process disclosed herein may also be employed for the examination and determination of potentially toxic substances or potential pharmaceutical agents or chemical or biological pesticides by examining their effect on nucleic acids or their amplifications in cellular or non-cellular systems.

The use of a cooling curve can be used to characterize all nucleic acids, including nucleic acid products resulting from many in vitro reactions, such as PCR, 35k, and TAS, as well as those nucleic acid products resulting from in vivo processes/reactions. It is suitable for analysis of serial or single reactions, and allows for simultaneous nucleic acid qualification/quantification. That is, the methods can be used qualitatively to detect the presence of a target nucleic acid, or can be used quantitatively (e.g., by inclusion of a control nucleic acid sequence of known quantity) to not only detect the presence of a nucleic acid of interest, but to determine the amount of target nucleic acid in a sample. The methods described herein can be used in genetic analyses, permitting, for instance, the detection of severe genetic diseases, such as cystic fibrosis, from traces of biopsy material or amniotic fluid. The methods described herein can be used to examine point mutations responsible for certain genetic diseases. The methods described herein can also be used in epidemiological studies on infectious and hereditary diseases.

Among the many optional features of the invention is the inclusion of an in vitro amplification reaction as an initial procedure for detection of target nucleic acids. According to the method employing these procedures, any suitable in vitro amplification reaction may be used. Typically, the amplification is performed using PCR or a process derived from it or based on it. In these embodiments, the amplified product is used as the target for detection (although it is to be recognized that the original nucleic acid may also participate in detection as well). In these embodiments, in general, the method can be characterized as follows: subsequent to the amplification reaction, at least one probe, which is detectable and capable of interacting with at least one amplified nucleic acid product, is placed in contact with amplification products and exposed to the action of a decreasing temperature gradient, wherein the initial temperature of the gradient is capable of at least partially denaturing nucleic acids, though full denaturation is preferable, with at least one measurable parameter undergoing variation through the action of the gradient. Like the basic method described above, the entire amplification and detection reaction, including qualitative and quantitative analysis using a cooling gradient, may be carried out in a reaction chamber/detection compartment, preferably without any opening of the compartment to the environment in general.

The process disclosed herein permits the amplification to be performed in a homogenous phase or on a solid phase, preferably using a primer that is attached to a solid phase and that has an extended sequence to which the labeled probe can hybridize. Thus, the concentration of the probe can be determined either specifically at the solid phase support or within the free solution. Preferably, at least one molecule of fluorescent dye is linked to a nucleic acid molecule, the sequence of which is identical or homologous to the nucleic acid to be detected or to the co-amplified nucleic acid standard. The amount of identity is not critical, so long as the probe sequence has a sufficiently high level of identity that it can detect the nucleic acid of interest. Preferably, the probe specifically detects the nucleic acid of interest; however, in embodiments, it may also detect other nucleic acids. In such a situation, the method may be used as a presumptive identification/detection, which can be followed by a more precise identification. Such a method would be useful in screening multiple samples for nucleic acids of interest—because the method is rapid and relatively inexpensive, use as an initial screening method could be economically feasible.

Thus, in some embodiments, a primer used for amplification is fixed to a surface, such as that of magnetic beads. For the purpose of amplification and hybridization, magnetic beads can be maintained in the form of a suspension by a magnetic field; for the purpose of laser fluorescence observation, however, during the reassociation process induced by the cooling temperature gradient, they may be withdrawn from the solution and fixed at a defined spot. Thus, the laser beam may be directed directly to the particle surface, and the fluorescence during reassociation of the probe may be monitored specifically. This process is particularly suited for use with a single melting domain, and permits use of oligomeric, non-intercalating fluorescent dyes as optical markers.

Once the nucleic acid molecule with the fluorescent dye linked thereto has been added to the reaction mixture after amplification has taken place, hybridization with the amplified nucleic acids is effected, preferably by effecting thermal denaturation followed with a subsequent cooling gradient during which renaturation occurs. However, among other options, it is also possible to add the nucleic acid molecule having the linked fluorescent dye to the reaction mixture before amplification has taken place. Here, the probe is to be added as a non-amplifiable double-stranded RNA or as a non-amplifiable chemically modified nucleic acid.

For nucleic acid amplification, one embodiment uses a primer of the primer pair employed for amplification, which primer contains a G:C-rich region at the 5′ terminus, for example from 15 to 20 G:C nucleotides. Extension of the primer incorporates the label into the target nucleic acid. In such situations, preferably, the label is a label that has an altered detectable property (e.g., fluorescence) when in the context of a single-stranded and a double-stranded nucleic acid molecule.

In an embodiment, the fluorescent probes are added subsequent to amplification. This means that initially, during the amplification reaction, the fluorescent probes (e.g., used for standardization (where a control reaction is run) and/or quantification) are stored spatially separated from the amplification process. Further, in some embodiments, the probes used for standardization differ in more than one position from the target nucleic acid being analyzed. In order to improve the signal/noise ratio in the subsequent determination using the employed probe, it is desirable not to add too little probe to the mixture to be amplified.

In one embodiment of the process according to the invention, the probe used for standardization and/or quantification and/or analysis of the target polynucleotide is a single-stranded oligo- or polynucleotide that, however, cannot participate in the amplification reaction because of chemical modification. Only by suitable manipulation following the amplification reaction, is the single-stranded probe exposed and capable of hybridizing with the corresponding nucleic acids being analyzed. Thus, for example, the probe, if present in the form of a single-stranded nucleic acid, may be inactivated in the form of a “hairpin structure” and may thus be prevented from participating in the amplification reaction.

The oligo- or polynucleotides to be used as probes in a particularly preferred fashion have one or more structural elements with at least two chemical substituents, each being capable of interacting with electromagnetic waves, with cleavage or linkage of stable bonds, or by absorption or emission of radiation. For a substituent that is particularly suitable for interacting with electromagnetic radiation, with cleavage and linkage of stable bonds, such as covalent bonds, psoralen or its derivatives have proved successful. For a structural element serving as an actual marker of the probe, luminescent dyes such as fluorescent dyes having high quantum yield such as dyes from the thiazole orange class have proved beneficial. Preferable are dyes having large Stokes shift which, dependent on hybridization condition, alter the luminescent properties.

It can be advantageous that the spectra of the structural elements at the respective sensitive sites which, on the one hand, are to be excited for cleavage and linkage of for instance, covalent bonds or, but on the other hand, are to be regarded as absorption or emission maxima, are far enough apart so that each excitation will not interfere with the function of the other structural element. Likewise, it is possible to combine both chemical structures in a single chemical structure if linkage and cleavage of bonds each occur at different wavelengths such as is possible with maximum fluorescence of this structure. Thus, the respective functions, probe fixation within a non-amplified structure on the one hand, and spectroscopic identification of the structure on the other hand, cannot interfere with each other. Where two separated structural elements have said separated functions, it is preferable that they should not fall below a distance of at least 10 nucleotides on the oligo- or polynucleotide strand.

In an embodiment of the process described herein, masked oligo- or polynucleotide probes are added to the above-described mixture of substances, the amplification reaction is carried out as described, and subsequently, the masked probe is released, by radiation for example, and hybridizes with the amplification product under analysis and, and the hybridized complex is detected by a time/temperature cooling gradient in homogenous solution. Using this embodiment, it is possible to perform the analysis in such a way that the hybridization labeled probe no longer has to be located separately within the compartment of the reaction chamber in order to add it to the reaction mixture immediately prior to the actual analysis. Thus, it is possible to provide the labeled probe with the other reagents in a form in which it cannot participate in the subsequent amplification process. Thereby, it is no longer necessary to separate amplification mixture and probe within the measuring chamber.

In one embodiment, in order to analyze a mixture of amplified nucleic acids, a time-controlled cooling temperature gradient is applied after addition of substances needed in the reaction, and the renaturation behavior of the nucleic acids is measured. This is done through the variation of spectroscopic parameters of the substance interacting with the nucleic acid. Variation of the spectroscopic parameter is monitored over time or in equivalent fashion as a function of temperature change. Evaluation of the function of variation in spectroscopic behavior of the substance interacting with the nucleic acid permits the determination of the presence or number or degree of homology of an examined nucleic acid with the corresponding standard. Preferably, evaluation of these data is done on-line using a data processing system.

One advantage of the invention is that amplification of nucleic acids and subsequent analytics can be carried out in a single sealable reaction compartment. As such, disposal of the reaction/detection materials, including potentially hazardous materials, without opening the compartments is possible, and a potential source of contamination is eliminated. Furthermore, such a procedure also permits storage of reaction vessels in sealed condition over prolonged periods of time so that archiving of the often valuable substances is made possible. Storage may be accomplished at any temperature; however, frozen storage is preferred. Likewise, the process according to the invention advantageously permits experiments to be repeatable, optionally at a later time even after prolonged interim storage, or the amplified mixture to be preparatively processable and analyzable.

The process according to the invention is particularly suitable for analyzing mixtures of substances, preferably nucleic acids, where at least one component in the temperature region of the time/temperature cooling gradient is subject to thermal conversion. By adding and co-amplifying a standard having precisely known number of copies the process of the invention can be standardized and permits quantitative statements about the amplified nucleic acids of the sample to be examined.

In another embodiment of the process disclosed herein, the nucleic acid renaturation process initiated by the decreasing temperature gradient is detected using wave length variation and/or shift in fluorescent intensity and/or variation in excited state lifetime, or using the principle of the so-called energy transfer (Forster Transfer), or via concentration effects, or using various, preferably hydrophobic, interactive properties of the labeled probe. More specifically, in embodiments, apart from direct fluorescence intensity measurement, a Forster Transfer between closely adjacent fluorophores or the parameter of fluorescence polarization may be used according to the invention. In another embodiment, a probe that is covalently linked, either internally or at the terminal ends, to one or more dye molecules is used in the cooling curve. Only when the probe is present in a fully hybridized homoduplex or heteroduplex, will a maximum fluorescence intensity from the fluorescent dye intercalated in the double-stranded molecule be obtained. The fluorescence intensity of the reaction batches conducted in parallel may be observed simultaneously by using a camera. The relative heights of the fluorescence variations may be converted directly to copy numbers by a computer connected to the detector.

The methods described herein permit simultaneous or sequential detection of multiple different amplified nucleic acids, or other target nucleic acids to be analyzed. This is effected by using a multiplicity of dyes, which may be distinguished from each other spectroscopically, and which permit the analysis of the various amplified nucleic acids and/or other target nucleic acids of interest, through which at least one independent calibrating substance is introduced. This, in particular, is possible where the various nucleic acids to be analyzed interact with differently labeled participants in hybridization.

Detection of the measuring signal is conducted, for example, by measuring the fluorescence generated by the dyes which, in particular, may be excited continuously or in pulses, such as by a laser or other generator of electromagnetism (e.g., UV light generator). Occasionally, intercalating dyes have another favorable property relating to the excited state half-life. In the case of ethidium bromide, the half-life of the excited state is greater by more than 10 fold if the fluorescent molecule is in the intercalated state. Thereby, a pulsed laser excitation may be used where in the subsequent phase of fluorescence emission, the fluorescence intensity can be measured without the influence of scattered light from light used for excitation. Using the process according to the invention, it is possible to employ dyes such as ethidium bromide, preferably at tow concentrations (preferably from 10⁻¹⁰ to 10⁻⁷ M).

An embodiment of the process disclosed herein is based on the premise that the amplified nucleic acids contain at least one co-amplified nucleic acid standard, the sequence of which is homologous to a sequence of one of the nucleic acids of interest to be analyzed and preferably identical to that sequence. In another embodiment, the nucleic acid standard may contain one or more point mutations which, in particular, lie in a sequence region of lowest stability. However, care must be taken that any point mutation lies outside the primer binding sites if enzymatic amplifications are performed. The nucleic acid standard may also be a natural component of the nucleic acid to be analyzed.

According to the process disclosed herein, it is also possible to observe successful amplification of a specific nucleic acid without adding a labeled standard fragment to the reaction batch after amplification has taken place. In this preferred procedure of the invention, specifically those primers required for amplification are employed which then hybridize at the corresponding sites in the sequence of the nucleic acids in question. However, the corresponding sequences between the primer sites may be so different that when passing the cooling temperature gradient, both sequences—the amplified test and standard sequence—renature separately, and preferably, renature in cooperative fashion. This feature allows use of sequences having a sequence deviation to such extent that heteroduplex formation is no longer possible. In such a situation, it is not necessary to add a labeled standard fragment after amplification has taken place. Different melting temperatures of both sequences may be influenced, for example, by greatly varying the length of the sequence or by selecting a poly-A/T sequence. The question of whether an amplification reaction has taken place may then be decided by employing the process described herein, for example, in a cooling temperature gradient with simultaneous presence of ethidium bromide, where quantitative detection is likewise possible.

Preferably, a fluorescent marker is used as the renaturation signal. Particularly suitable for this purpose are fluorescent dyes possessing the property of strongly fluorescing only when incorporated between base pairs (intercalation). If such dyes are incorporated into a double helix due to a renaturation process, this can be registered by a change in fluorescence intensity (increase in fluorescence). Where double helices have different stabilities as is the case with homoduplex and heteroduplex, the signal changes will occur at different temperatures and may be analyzed and evaluated separately. Furthermore, the precise renaturation temperature reflects possible differences in sequence as an indicator for so-called virus drifts, which may occur due to mutations.

As should be evident from the disclosure above, the method of the invention includes many embodiments. Among the non-limiting exemplary embodiments may be mentioned: a process for the qualitative and quantitative analysis of at least one in vitro amplified nucleic acid in a sample, in a reaction means comprising a reaction chamber, where the process includes the steps of: (i) including in the sample, during or subsequent to amplification of the nucleic acid, at least one probe that interacts with the nucleic acid to be detected, the probe being a) an oligo- or polynucleotide that hybridizes with the nucleic acid, b) a dye that intercalates with the nucleic acid, or c) a combination of the two, where the probe has a spectroscopically measurable parameter; (ii) exposing the sample to the action of a cooling gradient that, at least partially, renatures the amplified nucleic acid in the sample which exists in an at least partially denatured state at the start of the cooling curve, and that effects variation in the spectroscopically measurable parameter of the probe, creating a measurable signal; and (iii) detecting the measurable signal over one or more points of time through out the time course of the cooling temperature gradient. The spectroscopically measurable parameter of the probe can be at least one luminescent or fluorescent dye, and the probe can include a nucleic acid portion that interacts with the in vitro amplified nucleic acid during its renaturation accompanied by a change in the measurable signal. The spectroscopically measurable parameter can include a plurality of dyes distinguishable from each other spectroscopically, such as when excited by a laser or other energy (e.g., UV radiation).

The measurable signal can be detected, among other ways, using wavelength variation, shift in luminescence or fluorescence intensity, variation in fluorescence polarization, variation in excited state lifetime, or a combination thereof. It also can be detected, among other ways, using the principle of energy transfer or through a concentration effect.

In some embodiments, the reaction mixture includes at least one co-amplified nucleic acid standard, the sequence of which is homologous to a sequence to be analyzed, with the exception of at least one point mutation. Alternatively or in addition, the reaction mixture can include at least one co-amplified nucleic acid standard having a primer region, the sequence of which is homologous to the primer region of the amplified nucleic acid. The nucleic acid standard can be a natural component of the amplified nucleic acid, but is not limited as such.

Whether the amplification is carried out in free solution or using a primer attached to a solid phase, the amplified nucleic acid hybridizes with the probe, and the analysis is determined either attached to the solid phase or within the free solution. As such, the probe is at least one molecule of fluorescent dye linked to a nucleic acid molecule, the sequence of which is identical or homologous (partially identical) to the amplified nucleic acid to be detected or to a co-amplified nucleic acid standard. The fluorescent dye linked to the nucleic acid molecule can be added to the reaction mixture after completing amplification, and is hybridized with the denatured amplified nucleic acid during its subsequent renaturation under the cooling gradient. Alternatively, among other choices, the fluorescent dye linked to the nucleic acid molecule is added to the reaction mixture prior to completing amplification, and the probe is a non-amplifiable double-stranded RNA or a non-amplifiable chemically modified nucleic acid. A primer of a primer pair can be used for the amplification, where the primer encodes a G:C-rich region (e.g., of 2-5 nucleotides) at its 5′ terminus. In one embodiment, the primer has from 15 to 20 G:C residues at its 5′ terminus. The probe can be an oligo- or polynucleotide having at least two chemical structural elements, wherein (a) each chemical structural element can be detected, upon interacting with electromagnetic waves, by absorption or emission of radiation, and (b) one of the structural elements, upon interacting with electromagnetic waves, can link to another position on the oligo- or polynucleotide. Typically, but not always, the chemical structural elements have a chromophoric system or center, for example, one in which the chromophoric system luminesces via a dye substituent thereon. The chemical structural element that can link to another position on the oligo- or polynucleotide can be a photochemical crosslinker, and can include psoralin or a psoralin derivative. In some embodiments, the spacing between the two chemical structural elements is between 8 to 12 nucleotide positions, although this can vary outside of this range. Thus, the probe can be an oligo- or polynucleotide having at least one chemical structural element (a) having a stable bond that, upon interacting with electromagnetic waves, is capable of cleavage and subsequent linkage with the amplified nucleic acid and (b) that can be detected, upon interacting with electromagnetic waves, by absorption or emission of radiation, wherein said structural element is not a purine or pyrimidine substituent of naturally occurring nucleotide components.

As discussed below in more detail, the method can be practiced in a reaction vessel or chamber. In embodiments, the reagent mixtures for amplification and/or detection are stored in spatially separated matrices, and, subsequent to sealing the reaction chamber, are introduced into the reaction process. Regardless of the reaction vessel used, the temperature is varied during renaturation. In selected embodiments, the temperature at time T₀ is 100° C., 99° C., 98° C., 97° C., or 96° C. In some embodiments, the cooling gradient is a time-controlled decreasing temperature gradient, the variation of the spectroscopically measurable parameter is monitored as a function of time, temperature, or time and temperature, and this monitoring is analyzed by temperature gel electrophoresis, chromatography, or directly in homogenous solution, or a combination thereof. Alternatively, another non-limiting option is to use a data processing system to perform analysis. In these embodiments, in general, the presence, number, homology, or combination thereof of the amplified nucleic acid depends on the monitored spectroscopically measurable parameter. In another aspect of this embodiment, the analysis is effected by microtitration.

In one embodiment of the method of the invention, the reaction means includes (a) at least one multiple-well-containing sheet, each well being a reaction chamber that includes the probe and lyophilized amplification reagents, and (b) a sealing sheet cooperating with the multiple-well-containing sheet in a manner independently sealing each reaction chamber with a seal that becomes an interior surface of the reaction chamber. The reagents for amplification and/or detection can be present in at least one water-soluble matrix, which can include one or more than one stabilizer, and/or one or more than one sugar, such as a trehalose or saccharose. For example, the reagents in the reaction mixture can include amplification primers, buffer components, at least one polymerase, and co-factors.

In some embodiments, at least one reaction chamber of the well-containing sheet in which the method is performed includes a reagent/probe-containing matrix and the chamber interior surface of the corresponding seal includes hybridization reagents. In one embodiment, the reaction means is composed of kit systems. In one embodiment, the methods are carried out using computer-controlled, time-dependent regulation of the temperature of the reaction chamber. In one embodiment, the methods include optical-excitation-effecting emitting of a fluorescence signal and optical detection of the fluorescence signal. In one embodiment, the qualitative and quantitative analysis of the methods described herein can be accomplished without opening the reaction chamber, which may or may not be permanently sealed.

In another general aspect, the invention provides a device for performing the method of the invention. In essence, the device is a machine that is capable of controlling reaction parameters and providing information to the practitioner relating to reaction parameters and detection of signals from reaction mixtures. Typically, the device is capable of holding one or more reaction chambers for performing amplification and/or detection reactions (discussed in more detail below). The device is preferably provided as a single unit, but may comprise multiple units that are connected in a suitable way to allow communication of information from one unit to another (e.g., electrical cables, optical transmission/reception). While not limited in the means for providing information to the practitioner, typically, the device will comprise a video screen, monitor, panel, etc. for visual display of reaction parameters, and/or a printer for making paper copies of information of interest.

The device for performing the process disclosed herein has a means for time-dependent regulation of the temperature of the reaction chambers to be used in the process. In one embodiment, the time-dependent regulation of the temperature is controlled by a programmable unit. The read-out unit of the device (which may be understood as a detector) preferably consists of an optical unit capable of registering photons. Particularly preferred are such units which are suitable for registering emitted fluorescent light. Likewise contemplated is equipment capable of detecting other spectroscopic properties, such as nuclear spin or electron spin etc., which can be correlated to conformational changes of the nucleic acid double-helix or other structural variables, or the use of chromatographic procedures. Using the method of hydrophobic interaction chromatography, molecules having hydrophobic ligands, as represented by partially denaturing structures of the substances to be analyzed, may be separated from the duplexes.

The device for operating the process disclosed herein is capable of accommodating a system of reaction compartments, preferably a sheet system with ready-to-use reagents in freeze-dried form. Preferably, the reaction compartments are arranged in microtitration form. Preferably, the reagents of the means for operating the process are fixed and/or stored in at least one water-soluble matrix. Preferably, the matrix contains stabilizers such as sugars, particularly trehalose or saccharose. Preferably, the means for operating the process of the invention comprises reaction compartments and/or other reagent reservoirs, amplification primers, buffer components, and at least one polymerase and usual co-factors for performing the amplification reaction. In another preferred embodiment of the means for operating the process of the invention, the reaction chamber or reaction compartment is provided with an additional separate reagent reservoir in a matrix located within the sheet sealing the compartment. Here, preferably, the labeled probe with the buffer substances required for hybridization are stored.

Such a device may include for example Stratagene's Mx4000® Multiplex Quantitative PCR System, which is adapted (e.g., by including a computer program) such that the system is able to run, after an amplification reaction, a cooling curve in which the starting temperature can be as high as 100° C. As the temperature decreases the single strand nucleic acid product renatures to form a double stranded product by either a step-wise or continuous decrease in temperature, with fluorescence data being collected at various time points, such as at each step. When renaturation is performed in the presence of SYBR Green dye, the magnitude of the increase in fluorescence intensity of the SYBR Green dye due to its intercalation into dsDNA provides an indicator of the amount of renatured dsDNA at each point in the cooling curve. In thermal renaturation profiles for complex nucleic acid mixtures such as those generated during PCR reactions, two or more semi-discrete populations with different transition temperatures can typically be identified. Populations with a Tm of 80° C. or higher correspond to larger PCR products and can usually be assigned to the specific DNA product. DNA products displaying melting temperatures of <75° C. typically correspond to nonspecific DNA products. Exemplary fluorophores that can be used in the reaction (in conjunction with commercially available devices, such as the Mx4000 from Stratagene, include indocarbocyanine (C3), indodicarbocyanine (C5), Cy3, Cy3.5, Cy5, Cy5.5, Cy7, Texas Red, Pacific Blue, Oregon Green 488, Alexa Fluor 488, Alexa Fluor 532, Alexa Fluor 546, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodaminew), carboxy tetramethylrhodamine (TAMRAw), carboxy-X rhodamine (ROXm), LIZ, VIC, NED, PET, SYBR, PicoGreen, RiboGreen, and the like. Descriptions of fluorophores and their use can be found in, among other places, R. Haugland, “Handbook of Fluorescent Probes and Research Products”, (2002), Molecular Probes, Eugene, Oreg.; M. Schena, “Microarray Analysis” (2003), John Wiley & Sons, Hoboken, N.J.; Synthetic Medicinal Chemistry 2003/2004. Catalog, Berry and Associates, Ann Arbor, Mich.; G. Hermanson, Bioconjugate Techniques, Academic Press (1996); and Glen Research 2002 Catalog, Sterling, Va. Near-infrared dyes are expressly within the intended meaning of the terms fluorophore and fluorescent reporter group.

In yet another general aspect of the invention, articles of manufacture are provided. The articles find use in, among other things, performing nucleic acid amplification and detection methods. In general, the articles of manufacture comprise one or more reaction vessels or chambers for performing amplification reactions, detection assays, or both. The articles comprise at least one vessel wall that is suitable for transmission of a detectable signal to an external detector, such as an instrument that can detect electromagnetic radiation, particularly in the ultraviolet (UV) and visible (VIS) ranges (i.e., a UV-VIS detector). The shape and size of the reaction vessels are not critical to the article of manufacture, and can be varied to accommodate the needs or desires of the practitioner. For example, the vessels can be generally conically shaped, such as found in a microfuge tube, or generally cylindrical, such as found in some microtiter plates. The vessels can be designed to hold any volume, but will generally be designed to contain from 20 microliters (ul) to 1 milliliter (ml). While not limited in shape, size, or manner of construction, exemplary articles of manufacture according to the invention are multi-well microtiter plates, micro-centrifuge tubes (e.g., PCR reaction tubes), and relatively thin plastic sheets comprising multiple reaction/detection wells.

The articles of manufacture comprise one or more vessels or chambers for performing detection assays according to the invention. As discussed above, preferably, the detection assay is performed in the same reaction vessel as an amplification reaction, and results in production of a detectable signal corresponding to a target nucleic acid. Thus, to allow for detection, at least one region of the a vessel wall should be capable of transmitting a detectable signal from the interior of the vessel to the exterior, where it can be detected. Thus, the reaction vessel will comprise, at least as part of a portion of one surface, a material that is capable of transmitting a detectable signal generated inside the vessel.

In embodiments, the articles of manufacture comprise a two-part vessel: the first part defining a reaction chamber and the second part defining a top, lid, or cap. In these embodiments, the top may be provided as an integral part of the vessel, such as by way of a hinged connection to the remainder of the vessel (e.g., as in a flip-top cap of a microfuge tube). Alternatively, it may be provided as a separate unit that is attached or otherwise connected to the remaining portions of the vessel at an appropriate time. For example, it may be a screw-cap, plate lid, or a plastic sheet that is place over the reaction vessel container portion after all reagents, samples, etc. have been placed inside the vessel. It is to be noted that the cap, lid, etc. may be attached to the rest of the vessel in a permanent manner (e.g., by adhesive, sonic welding, heat welding, etc.) or in a removable manner (e.g., screw cap, flip top).

One particular embodiment of the article of manufacture is a multi-well (multi-vessel) sheet structure. According to this embodiment, multiple hollow pockets or recesses serving as reaction chambers (vessels, compartments) are provided on a relatively thin sheet of plastic material. A second sheet of plastic material is also provided to serve as a top surface for the chambers. The two sheets may be provided as a single unit (i.e., connected at some point) or as two separate units to be combined into a final product. Preferably, one or both of the sheets are thermally weldable and suited to accommodate ready-for-use reagent mixtures in freeze-dried or matrix-bound form. The sheet materials may be any thermoplastic or thermosetting resins, or combinations thereof, but should be selected in conjunction with the particular detectable label so as to ensure that the signal from the label can transmit through the selected plastic. In other words, the practitioner should select the sheet material such that direct optical measurement of the reaction chamber contents is possible. Hence, the sheet material is typically translucent or transparent, at least for specific wavelength regions of electromagnetic radiation.

Preferably, some or all of the reagents needed to perform the process according to the invention are stored in spatially separated matrices located on the sheet materials (either the top, bottom, or both), and subsequent to sealing the reaction chamber, are introduced into the reaction process. Preferably, the reaction chambers of the sheet material are separated from each other at a distance that is similar or identical to the holes found in a commercially available microtiter plate. This configuration has the advantage that equipment suitable for processing microtitration plates may be used in the technology described herein.

As discussed above, reaction and analysis may be carried out in a single reaction compartment. Preferably, an article of manufacture having the general configuration of a microtiter plate is used, which permits 96 samples or portions of 96 samples to be analyzed at one time. Preferably, the microtiter wells can hold reaction volumes of from 20 ul to 100 ul. Though single reaction vessels may be used, sheets having a configuration of a standard microtiter plate for use in commercially available in vitro amplification devices are preferably used, which have multiple wells or recesses accommodating the samples, and which are able to be thermally regulated and allow for detection of ultraviolet and/or visible light and/or of fluorescence signals of commercially available fluorescent dyes. The sheets may be charged with some or all of the generally required reagents for amplification reactions (enzymes, primers, buffers, stabilizers, etc.) and preserved for long periods of time in lyophilized condition. Preferably, trehalose or saccharose is used as a stabilizer.

In use, following addition of the samples to be analyzed, the reaction vessels/sheets may be closed with a top sheet for covering the vessels/sheet comprising the reaction wells. Reagents may be fixed or placed in compartments which are not available to participate in the reaction process at the beginning, such as a specifically labeled probe which is lyophilized and stabilized in a buffer mixture and is required subsequent to amplification reaction for hybridizing to the amplification product and the labeled probe. Subsequent to sealing the reaction compartments, the amplification reaction takes place at a static temperature or in a thermocycler as a polymerase chain reaction (PCR) (see, for example, 3SR, Self-sustained Sequence Replication; TAS, Transcription based amplification system; J. C. Guatelli et al., Proc. Natl. Acad. Sci. 87:874-1878 (1990); Kwoh, D. Y., Davis, O. R., Whitfield, K. M., Chapelle, H. L., Dimichele, L. J. and Gingeras, T. R. Proc. Natl. Acad. Sci. U.S.A. 86:1173-1177, (1989)). In the amplification step, a control/standard (if present) and target template are amplified at a constant ratio so that the reaction final product preferably contains from 100 nanograms (ng) to 1 ug of amplified nucleic acid. After the amplification step, the specialized label probe is contacted with the reaction product(s), and subjected to a cooling curve, allowing the characterization of the reaction product(s).

In another embodiment, the reaction compartment includes a luminescent dye, preferably a fluorescent dye, preferably having intercalating properties, binding at multiple positions in double-helical structures and having modified spectroscopic properties in the bound state. When in the analysis following amplification using the time/temperature cooling gradient, at the beginning of the cooling gradient, the corresponding double-stranded structures are completely denatured, and the dye is not associated with the denatured nucleic acid. The process of renaturation through the cooling curve over time is recorded spectroscopically. In the process, the concentration of the free dye is chosen such that it is greater than the number of free binding sites. It is to be noted that the double-stranded structures analyzed by the cooling method are not limited to those produced in an amplification reaction, and include any structure with at least one double stranded component.

In yet a further general aspect of the invention, kits are provided. The methods described herein provide for manufacturing of test kits, and provide for automated analyses. The methods and kits described herein may be used in the fields of microbiology, human genetics, phytoanalytics, forensic analytics, and include screening of target substances for active agents, which may be evaluated through DNA or RNA amplification or modification, as well as simple toxicity assays. While not limited in use, the kits can advantageously be used for amplification and/or detection of target nucleic acids. In general, the kits comprise at least two articles of manufacture according to the invention, in packaged combination. The kits may also comprise some or all of the reagents, solutions, etc. needed to perform a method according to the invention. As detailed below, in some embodiments, the kits of the invention comprise all of the reagents needed to detect a target nucleic acid sequence, and use of the kits merely requires addition of a sample suspected of containing the target nucleic acid.

In embodiments, the kits comprise multiple sheets for performing amplification and detection methods. As such, the kits comprise two or more sheets comprising reaction vessels with storable and directly usable reagent mixtures, where it is merely necessary to charge the reaction vessels with the sample to be analyzed which, in a sealed condition, is then subjected to an amplification procedure and subsequent analysis.

In one embodiment, the kits comprise two or more multi-well structures in a microtiter plate configuration, where located in the wells of the microtiter plate are some, preferably all, of the reagents required for specific amplification of a nucleic acid. For example, the reagents, including probe(s), can be provided in lyophilized form within or attached to the walls of the reaction vessels. In use, the sample to be analyzed, for example, in the form of an aqueous solution, is added prior to the amplification reaction. Subsequently, the reaction compartments are sealed with a second or sealing sheet, such as one that forms the lid of the reaction compartment, with the second sheet containing at least one further matrix with reagents not participating in the actual amplification reaction. The sheet is positioned in a thermostat block in order to carry out the enzymatic amplification reaction. Here, amplifications may be performed both at homogenous temperature and in periodically varying temperature cycles (PCR). Subsequent to amplification, the reaction mixture is contacted with the second reagent reservoir, for example by inversion and thus wetting of the stored matrix, or by physical disruption of the matrix (e.g., sonic vibration, heating to degrade matrix components, etc.) and a homogenous solution is prepared. Following performance of a denaturation/renaturation process, an optical detection system records a detectable signal from the mixture, e.g., a laser-induced luminescence (fluorescence, phosphorescence), as a function of a linear temperature cooling gradient, which is time-controlled, such as by a thermoblock of a PCR machine. In embodiments, the initial temperature may be, for example, as high as about 100° C., 99° C., 98° C., 97° C., 96° C., or 95° C., and the final temperature as low as about 4° C. In another embodiment, the initial temperature is as high as is necessary to achieve at least partial denaturation of the target nucleic acid and its probe.

All patents, patent applications, and published references cited herein are hereby incorporated by reference in their entirety. While this invention has been particularly shown and described with references to preferred embodiments thereof, it is to be understood that various changes in form and details maybe made without departing from the scope of the invention encompassed by the appended claims. The spirit and scope of the present invention are not limited to the above embodiments, but are encompassed by the following claims.

1. A method for detecting a nucleic acid of interest, said method comprising:
forming a composition comprising one or more nucleic acids and one or more detectable labels, under conditions where at least some of the nucleic acids exist in a single-stranded state;
decreasing the temperature of the composition to allow the detectable label(s) and nucleic acid(s) to stably contact each other to form complexes; and
detecting the complexes,
wherein detection of complexes is indicative of the presence of the nucleic acid of interest.

2. The method of claim 1, wherein the nucleic acid of interest is a product of an in vitro amplification reaction.

3. The method of claim 2, wherein the amplification reaction is the polymerase chain reaction or a reaction derived from it.

4. The method of claim 2, wherein the amplification reaction and the step of detecting are performed in the same single vessel without the vessel being opened between the two reactions.

5. The method of claim 2, wherein the amplification reaction is performed using a primer attached to a solid phase.

6. The method of claim 2, wherein the step of decreasing the temperature of the composition is performed in a thermocycler, which is the same thermocycler used for the amplification, and wherein decreasing of the temperature occurs without removing the vessel from the thermocycler.

7. The method of claim 1, wherein the detectable label is an oligonucleotide comprising moiety that produces a detectable signal.

8. The method of claim 1, wherein the moiety that produces a detectable signal comprises a chromophore center that emits a detectable signal when the label is intercalated within double stranded nucleic acids.

9. The method of claim 1, wherein the detectable label comprises a chromophore center that emits a detectable signal when the label is intercalated within double stranded nucleic acids.

10. The method of claim 1, wherein formation of the complexes changes a spectroscopically detectable signal emitted by the label.

11. The method of claim 1, comprising using multiple labels to detect multiple target nucleic acid sequences.

12. The method of claim 1, further comprising amplifying a nucleic acid standard, which comprises a homologous, but not identical, sequence as the target nucleic acid.

13. The method of claim 1, wherein the step of detecting the complexes comprises detecting a signal emitted from the label upon excitation by a laser.

14. The method of claim 1, wherein the method is a quantitative method of detecting a target nucleic acid.

15. An article of manufacture comprising:
at least one chamber for performing an in vitro nucleic acid amplification reaction and an in vitro nucleic acid detection assay, wherein the chamber comprises:
at least one wall comprising an interior and exterior surface, wherein the interior surface comprises some or all of the reagents for detection of a nucleic acid of interest.

16. The article of manufacture of claim 15, wherein the article comprises multiple chambers that comprise a reaction vessel containment portion and a cap portion, and wherein all of the reagents for detection of the nucleic acid of interest are present on the cap portion.

17. The article of manufacture of claim 16, wherein the cap portion is permanently sealed to the containment portion.

18. The article of manufacture of claim 15, wherein the article is configured as a microtiter plate having 96 reaction/detection chambers.

19. The article of manufacture of claim 18, wherein at least a portion of a wall is capable of transmitting a detectable signal from the interior surface to the exterior surface.

20. A kit comprising, in packaged combination, two or more articles of manufacture according to claim 15.
